(19) United States
(12) Patent Application Publication
(10) Pub. No.: US 2026/0158090 A1
(43) Pub. Date: Jun. 11, 2026

(54) PRODUCTS TO TREAT AND/OR PREVENT MENSTRUAL SYMPTOMS

(71) Applicant: SOCIETE DES PRODUITS NESTLE S.A., Vevey (CH)

(72) Inventors: JUSTINE PETIT, Lausanne (CH); CLARISSE MESLIER-AUDEBERT, Lausanne (CH); ANNE PETERSEN, Vevey (CH); MARJORIE GUITARD ULDRY, Savigny (CH)

(21) Appl. No.: 18/703,803

(22) PCT Filed: Oct. 21, 2022
(86) PCT No.: PCT/EP2022/079441

§ 371 Date: Apr. 23, 2024

(30) Foreign Application Priority Data
Oct. 27, 2021 (EP) 21205112.2

(51) Int. Cl. A61K 36/235; A23G 3/48; A61K 9/00; A61K 36/28; A61K 36/53; A61K 36/9068; A61P 15/00

(52) U.S. Cl. CPC A61K 36/235; A23G 3/48; A61K 9/0056; A61K 36/28; A61K 36/53; A61K 36/9068; A61P 15/00; A61K 2236/51

(57) ABSTRACT

A composition for use in treating and/or preventing menstrual symptoms wherein the composition comprises (i) fennel and chamomile or (ii) fennel, ginger and lemon balm.

### FIELD OF THE INVENTION

The present invention relates to compositions for use in treating and/or preventing menstrual symptoms. In particular, the present invention relates to confectionery products comprising botanicals and the use of said products to treat and/or prevent menstrual symptoms.

### BACKGROUND TO THE INVENTION

Menstrual symptoms have an undesirable impact on a notable proportion of woman. It is estimated that 60-85% of women are affected by (pre)menstrual symptoms, which may lead to productivity loss, recurring pain and absenteeism at school. Premenstruation may be associated with mood-related symptoms, bloating and discomfort. Menstruation may be associated with cramps and fatigue.

Pharmaceutical-based approaches to alleviate (pre)menstrual symptoms may be associated with side-effects. In addition, it is recognised that alternative, non-pharmaceutical approaches may be preferred by individuals. In this regard, various botanicals have been reported to alleviate one or more (pre)menstrual symptoms.

There is thus a need for compositions and products that are effective at alleviating (pre)menstrual symptoms, in particular as alternatives to pharmaceutical-based approaches.

### SUMMARY OF THE INVENTION

The present invention relates to a composition comprising botanicals for use in treating and/or preventing one or more (pre)menstrual symptoms. In particular, the composition may be a confectionery product as provided herein.

In one aspect, the premenstrual symptom is fatigue. In one aspect, the premenstrual symptom is mood swings. In one aspect, the premenstrual symptom is binge eating or cravings. In one aspect, the premenstrual symptom is headache. In one aspect, the premenstrual symptom is abdominal cramp.

Accordingly, in one aspect the present invention provides a composition for use in treating and/or preventing menstrual symptoms wherein the composition comprises fennel and chamomile.

In one aspect, the menstrual symptom is abdominal cramp. In one aspect, the menstrual symptom is fatigue. In one aspect, the menstrual symptom is mood swings. In one aspect, the menstrual symptom is bloated feeling. In one aspect, the menstrual symptom is back pains.

The invention also provides a composition for use in treating and/or preventing menstrual symptoms wherein the composition comprises fennel, ginger and lemon balm.

The invention further comprises a combination of a first composition comprising fennel and chamomile and a second composition comprising fennel, ginger and lemon balm for use in treating and/or preventing menstrual symptoms.

In another aspect, the present invention relates to a method of treating and/or preventing menstrual symptoms comprising administering a composition comprising fennel and chamomile to a subject.

The invention further provides a method of treating and/or preventing menstrual symptoms comprising administering a composition comprising fennel, ginger and lemon balm to a subject.

The invention also relates to use of a combination of fennel and chamomile in the manufacture of a medicament for treating and/or preventing menstrual symptoms.

The invention further provides use of a combination of fennel, ginger and lemon balm in the manufacture of a medicament for treating and/or preventing menstrual symptoms.

The invention also provides a product, preferably a confectionery product, comprising fennel and chamomile.

The invention further provides a product, preferably a confectionery product, comprising fennel, ginger and lemon balm.

The invention also provides a kit comprising a first product, preferably a confectionery product, comprising fennel and chamomile and a second product, preferably a confectionery product, comprising fennel, ginger and lemon balm.

The invention further provides a process for the preparation of a confectionery product according to invention, comprising the steps of: a) providing a powder mix comprising (i) fennel extract powder or dried fennel powder and chamomile extract powder or dried chamomile powder; or (ii) fennel extract powder or dried fennel powder, ginger extract powder or dried ginger powder and lemon balm extract powder or dried lemon balm powder; b) combining the powder mix with a dough mix and mixing to form a combined dough; and c) moulding or shaping the combined dough to form a confectionery mass.

Suitably, other botanicals may be used as alternatives to those described herein. By way of example, cinnamon may be provided as an alternative to ginger; and sage may be provided as an alternative to chamomile and/or lemon balm. Raspberry leaf may also be provided as an alternative botanical which can relieve menstrual cramps. Accordingly, the compositions for use or products of the present invention may comprise an alternative botanical—such as one or more of those described above.

### BRIEF DESCRIPTION OF THE FIGURES

FIG. 1: A schematic demonstrating an illustrative process for producing a confectionery product of the invention.

FIGS. 2A-2B: Graphs depicting the severity and improvement/worsening of the pre-menstrual phase symptom of fatigue before and after consumption of a confectionery product of the invention.

FIGS. 3A-3B: Graphs depicting the severity and improvement/worsening of the pre-menstrual phase symptom of mood swings before and after consumption of a confectionery product of the invention.

FIGS. 4A-4B: Graphs depicting the severity and improvement/worsening of the pre-menstrual phase symptom of binge eating/cravings before and after consumption of a confectionery product of the invention.

FIGS. 5A-5B: Graphs depicting the severity and improvement/worsening of the pre-menstrual phase symptom of headache before and after consumption of a confectionery product of the invention.

FIGS. 6A-6B: Graphs depicting the severity and improvement/worsening of the pre-menstrual phase symptom of abdominal cramps before and after consumption of a confectionery product of the invention.

FIGS. 7A-7B: Graphs depicting the severity and improvement/worsening of the pre-menstrual phase symptom of bloating before and after consumption of a confectionery product of the invention.

FIGS. 8A-8B: Graphs depicting the severity and improvement/worsening of the menstrual phase symptom of abdominal cramps before and after consumption of a confectionery product of the invention.

FIGS. 9A-9B: Graphs depicting the severity and improvement/worsening of the menstrual phase symptom of fatigue before and after consumption of a confectionery product of the invention.

FIGS. 10A-10B: Graphs depicting the severity and improvement/worsening of the menstrual phase symptom of mood swings before and after consumption of a confectionery product of the invention.

FIGS. 11A-11B: Graphs depicting the severity and improvement/worsening of the menstrual phase symptom of bloating before and after consumption of a confectionery product of the invention.

FIGS. 12A-12B: Graphs depicting the severity and improvement/worsening of the menstrual phase symptom of back pain before and after consumption of a confectionery product of the invention.

FIGS. 13A-13B: Graphs depicting the severity and improvement/worsening of the menstrual phase symptom of binge eating/cravings before and after consumption of a confectionery product of the invention.

### DETAILED DESCRIPTION OF THE INVENTION

The present invention relates to compositions comprising botanicals for use in treating and/or preventing one or more (pre)menstrual symptoms.

### Menstruation

Menstruation is the regular discharge of blood and mucosal tissue from the inner lining of the uterus through the vagina. The menstrual cycle is characterized by the rise and fall of hormones. Menstruation is triggered by falling progesterone levels and is a sign that pregnancy has not occurred.

Symptoms in advance of menstruation that interfere with normal life may be referred to as premenstrual syndrome (PMS). These include acne, tender breasts, bloating, feeling tired, irritability, and mood changes. Other symptoms some women experience include painful periods and heavy bleeding during menstruation and abnormal bleeding at any time during the menstrual cycle.

Premenstruation may refer to a period of time before onset of menses. For example, premenstruation may refer up to 7 days, up to 5 days or up to 3 days before onset of menses. Premenstruation may be associated with mood-related symptoms, bloating and discomfort. Premenstrual symptoms may be assessed (e.g. before and after administration of the present composition or product) using a Premenstrual Symptom Screening Tool.

Menstruation may refer to the period of time following onset of menses. Menstruation may be associated with symptoms such as cramps and fatigue. Menstrual symptoms may be assessed (e.g. before and after administration of the present composition or product) using a Pain Visual Analogue Scale (PVAS) or a numerical scale for pain.

Premenstrual and/or menstrual symptoms may be assessed based on health related quality of life, which may be assessed using a SF36 questionnaire

(www.rand.org/health-care/surveys_tools/mos/36-item-short-form).

The composition for use or product of the present invention may reduce one or more premenstrual or menstrual symptoms. The premenstrual or menstrual symptom(s), and level of symptoms, may be determined using a method as described herein.

In one aspect, the premenstrual symptom is fatigue. In one aspect, the premenstrual symptom is mood swings. In one aspect, the premenstrual symptom is binge eating or cravings. In one aspect, the premenstrual symptom is headache. In one aspect, the premenstrual symptom is abdominal cramp.

In one aspect, the menstrual symptom is abdominal cramp. In one aspect, the menstrual symptom is fatigue. In one aspect, the menstrual symptom is mood swings. In one aspect, the menstrual symptom is bloated feeling. In one aspect, the menstrual symptom is back pains.

The composition for use or product may reduce one or more behavioural, psychological and/or physical premenstrual symptom. In particular, a composition for use or product comprising fennel and chamomile as provided herein may reduce one or more behavioural, psychological and/or physical premenstrual symptom.

The composition or product may reduce one or more behavioural, psychological and/or physical menstrual symptom. In particular, a composition for use or product comprising fennel, ginger and lemon balm as provided herein may reduce one or more behavioural, psychological and/or physical premenstrual symptom.

The combination of compositions for use or products described herein may reduce one or more behavioural, psychological and/or physical premenstrual and menstrual symptom.

### Fennel

Fennel (Foeniculum Vulgare) is a perennial, pleasant-smelling herb with yellow flowers which has been used as a medicament. ‘Fennel’ as used herein preferably refers to the fruit/seed (i.e. whole cremocarp and mericarp).

The fennel for use in the present invention may be obtained from any part of the fennel plant, e.g. from the fennel seeds. Suitably, the fennel for use in the present invention is dried fennel seeds, for example in the form of a powder. Suitably, the fennel for use in the present invention may be in the form of a fennel extract, for example a fennel extract powder. Suitably, the fennel extract may be a fennel seed extract. The fennel extract may be a food grade extract.

Fennel (e.g. dry ground fennel seeds) may, for example, be extracted with water, ethanol, methanol, and/or glycerol as solvents or with supercritical carbon dioxide. Preferably, the fennel for use in the present invention is an aqueous extract of fennel seeds. Suitable processes for preparing fennel extracts are described in e.g. Angelov, G. and Boyadzhieva, S., 2016. Chemical and Biochemical Engineering Quarterly, 30(2), pp. 245-253 and Damjanović, B., et al., 2005. Food Chemistry, 92(1), pp. 143-149.

Preferably, the fennel for use in the present is a fennel extract powder. A fennel extract may be dried (e.g. spray-dried or lyophilised) to obtain a powder form. Suitably, the fennel extract is spray-dried. Any suitable carrier may be used for spray-drying (e.g. maltodextrin).

The composition for use according to the present invention may be administered to a subject to provide a daily dosage of 0.05 g-2.0 g fennel extract or dried fennel powder. Suitably, the composition may provide a daily dosage of 0.05 g-2.0 g, 0.05 g-1.0 g, 0.1 g-1.0 g, 0.1-0.5 g, 0.1-0.4 g, 0.1-0.3 g or 0.2-0.3 g fennel extract or dried fennel powder.

Preferably, the composition may be administered to a subject to provide a daily dosage of about 0.2 g to about 0.3 g of fennel extract or dried fennel powder.

Suitably, the composition may be administered to a subject to provide a daily dosage of about 0.20 g, about 0.21 g, about 0.22 g, about 0.23 g, about 0.24 g, about 0.25 g, about 0.26 g, about 0.27 g, about 0.28 g, about 0.29 g, about 0.30 g of fennel extract or dried fennel powder. Suitably, the composition may be administered to a subject to provide a daily dosage of about 0.25 g fennel extract or dried fennel powder.

### Chamomile

Chamomile is a member of Asteraceae/Compositae family and represented by two common varieties viz. German Chamomile (Chamomilla recutita or Matricaria chamomilla) and Roman Chamomile (Chamaemelum nobile). Suitably, the chamomile used in the present invention is German chamomile. The dried flowers of chamomile contain terpenoids and flavonoids. Chamomile preparations have been described to be used for ailments such as hay fever, inflammation, muscle spasms, menstrual disorders, insomnia, ulcers, wounds, gastrointestinal disorders, rheumatic pain, and hemorrhoids.

The chamomile for use in the present invention may be obtained from any part of the chamomile plant e.g. from the chamomile flowers. Suitably, the chamomile for use in the present invention is dried chamomile flowers, for example in the form of a powder. Suitably, the chamomile for use in the present invention may be a chamomile extract, for example a chamomile extract powder. Suitably, the chamomile extract may be a chamomile flower extract. The chamomile extract may be a food grade extract.

Chamomile (e.g. dry ground flowers of chamomile) may, for example, be extracted with water, ethanol, methanol and/or glycerol as solvents or with supercritical carbon dioxide. Preferably, the chamomile for use in the present invention is an aqueous extract of chamomile flowers. Suitable processes for preparing chamomile extracts are described in e.g. Z̆labur JS̆, et al. Molecules. 2020 Jan; 25(4):810 and Scalia, S., et al., 1999. Journal of pharmaceutical and biomedical analysis, 21(3), pp. 549-558.

Preferably, the chamomile for use in the present is a chamomile extract powder. A chamomile extract may be dried (e.g. spray-dried or lyophilised) to obtain a powder form. Suitably, the chamomile extract is spray-dried. Any suitable carrier may be used for spray-drying (e.g. maltodextrin).

The composition for use according to the present invention may be administered to a subject to provide a daily dosage about 0.1 g-5.0 g chamomile extract or dried chamomile powder. Suitably, the composition may provide a daily dosage of 0.1 g-5.0 g, 0.1 g-4.0 g, 0.2-4.0 g, 0.5-4.0 g, 0.5-3.0 g, 0.5-2.0 g or 0.5-1.0 g chamomile extract or dried chamomile powder. Preferably, the composition may provide a daily dosage of about 0.5 g to about 2.0 g chamomile extract. Suitably, the composition may provide a daily dosage of about 0.50 g, about 0.60 g, about 0.70 g, about 0.80 g, about 0.90 g, about 1.0 g of chamomile extract or dried chamomile powder. Suitably, the composition may provide a daily dosage of about 0.85 g chamomile extract or dried chamomile powder.

### Ginger

Ginger (Zingiber officinale) is a flowering plant whose rhizome, ginger root, is widely used as a spice and a medicine.

The ginger for use in the present invention may be obtained from any part of the ginger plant, e.g. from the ginger root. Suitably, the ginger for use in the present invention is dried ginger root, for example in the form of a powder. Suitably, the ginger for use in the present invention may be a ginger extract, for example a ginger extract powder. Suitably, the ginger extract may be a ginger root extract. The ginger extract may be a food grade extract.

Ginger (e.g. dry ground ginger root) may, for example, be extracted with water, ethanol, methanol and/or glycerol as solvents or with supercritical carbon dioxide. Preferably, the ginger for use in the present invention is an aqueous extract of ginger root. Suitable processes for preparing ginger extracts are described in e.g. Prakash, J., 2010. Journal of Medicinal Plants Research, 4(24), pp. 2674-2679 and Mesomo, M.C., et al., 2013. The Journal of Supercritical Fluids, 80, pp. 44-49.

Preferably, the ginger for use in the present is a ginger extract powder. A ginger extract may be dried (e.g. spray-dried or lyophilised) to obtain a powder form. Suitably, the ginger extract is spray-dried. Any suitable carrier may be used for spray-drying (e.g. maltodextrin).

The composition for use according to the present invention may be administered to a subject to provide a daily dosage about 0.1 g-5.0 g ginger extract or dried ginger powder. Suitably, the composition may provide a daily dosage of 0.1 g-5.0 g, 0.1 g-4.0 g, 0.2-4.0 g, 0.5-4.0 g, 0.5-3.0 g, 0.5-2.0 g or 0.5-2.0 g ginger extract or dried ginger powder. Preferably, the composition may provide a daily dosage of about 0.5 g to about 1.0 g of ginger extract or dried ginger powder. Suitably, the composition may provide a daily dosage of about 0.50 g, about 0.60 g, about 0.70 g, about 0.80 g, about 0.90 g, about 1.0 g of ginger extract or dried ginger powder. Suitably, the composition may provide a daily dosage of about 0.9 g ginger extract or dried ginger powder.

### Lemon Balm

Lemon balm (Melissa officinalis) is a perennial herbaceous plant in the mint family. The tea of lemon balm, the essential oil, and the extract are used in traditional and alternative medicine, including aromatherapy.

The lemon balm for use in the present invention may be obtained from any part of the lemon balm plant e.g. from the lemon balm leaves. Suitably, the lemon balm for use in the present invention is dried lemon balm leaves, for example in the form of a powder. Suitably, the lemon balm for use in the present invention may be a lemon balm extract, for example a lemon balm extract powder. Suitably, the lemon balm extract may be a lemon balm leaves extract. The lemon balm extract may be a food grade extract.

Lemon balm (e.g. dry ground lemon balm leaves) may, for example, be extracted with water, ethanol, methanol and/or glycerol as solvents or with supercritical carbon dioxide. Preferably, the lemon balm for use in the present invention is an aqueous extract of lemon balm leaves. Suitable processes for preparing lemon balm extracts are described in e.g. Arceusz, A., et al., 2015. Natural product communications, 10(6), pp. 977-981. Bogdanovic, A., et al., 2016. The Journal of Supercritical Fluids, 107, pp. 234-242.

Preferably, the lemon balm for use in the present is a lemon balm extract powder. A lemon balm extract may be dried (e.g. spray-dried or lyophilised) to obtain a powder form. Suitably, the lemon balm extract is spray-dried. Any suitable carrier may be used for spray-drying (e.g. maltodextrin).

The composition for use according to the present invention may be administered to a subject to provide a daily dosage about 0.1 g-5.0 g lemon balm extract or dried lemon balm powder. Suitably, the composition may provide a daily dosage of 0.1 g-5.0 g, 0.1 g-4.0 g, 0.2-4.0 g, 0.5-4.0 g, 0.5-3.0 g, 0.5-2.0 g or 0.5-2.0 g lemon balm extract or dried lemon balm powder. Preferably, the composition may provide a daily dosage of about 0.5 g to about 1.0 g of lemon balm extract or dried lemon balm powder. Suitably, the composition may provide a daily dosage of about 0.50 g, about 0.60 g, about 0.70 g, about 0.80 g, about 0.90 g, about 1.0 g of lemon balm extract or dried lemon balm powder. Suitably, the composition may provide a daily dosage of about 0.9 g lemon balm extract or dried lemon balm powder.

### Magnesium

Magnesium has been reported to reduce anxiety and/or depression.

Suitably, the magnesium is food grade magnesium.

The composition for use according to the present invention may be administered to a subject to provide a daily dosage of about 10-460 mg, 10-400 mg, 10-300 mg, 25-300 mg, 25-200 mg, 50-200 mg or 50-100 mg magnesium. Suitably, the composition may provide a daily dosage of about 50 mg, about 70 mg or about 100 mg magnesium. Suitably, the composition may provide a daily dosage of about 70 mg magnesium.

### Iron

The composition for use according to the present invention may be administered to a subject to provide a daily dosage of about 1-20 mg, 1-10 mg, 1-5 mg, 1-4 mg, 1-3 mg, or 2-3mg iron.

Suitably, the composition may provide a daily dosage of about 2.0 mg, about 2.2 mg, about 2.4 mg, about 2.6 mg, about 2.8 mg or about 3.0 mg iron. Suitably, the composition may provide a daily dosage of about 2.6 mg or about 2.8 mg iron.

Suitably, the iron is food grade iron.

### Composition

The composition for use according to the present invention may be administered to a subject to provide a daily dosage of about 0.1-0.3 g fennel extract or dried fennel powder and about 0.5-2.0 g chamomile extract or dried chamomile powder. The composition may be administered to provide a daily dosage of about 0.1-0.3 g fennel extract or dried fennel powder, about 0.5-1.0 g chamomile extract or dried chamomile powder and about 35-100 mg magnesium. The composition may be administered to provide a daily dosage of about 0.25 g fennel extract or dried fennel powder, and about 0.85 g chamomile extract or dried chamomile powder. The composition may be administered to provide a daily dosage of about 0.25 g fennel extract or dried fennel powder, about 0.85 g chamomile extract or dried chamomile powder and about 70 mg magnesium. Preferably, the fennel is fennel extract. Preferably the chamomile is chamomile extract.

The composition for use according to the present invention may be administered to a subject to provide a daily dosage of about 0.1-0.3 g fennel extract or dried fennel powder, about 0.25 g-2.0 g ginger extract or dried ginger powder and about 0.25 g-2.0 g lemon balm extract or dried lemon balm powder. The composition may be administered to provide a daily dosage of about 0.25 g fennel extract or dried fennel powder, about 0.9 g ginger extract or dried ginger powder and about 0.9 g lemon balm extract or dried lemon balm powder.

Preferably, the fennel is fennel extract. Preferably the chamomile is chamomile extract. Preferably the lemon balm is lemon balm extract.

The present invention also relates to the use of a composition comprising fennel and chamomile for reducing one or more (pre)menstrual symptoms. The composition may be a product comprising fennel and chamomile as provided herein.

In one aspect, the premenstrual symptom is fatigue. In one aspect, the premenstrual symptom is mood swings. In one aspect, the premenstrual symptom is binge eating or cravings. In one aspect, the premenstrual symptom is headache. In one aspect, the premenstrual symptom is abdominal cramp.

The present invention also relates to the use of a composition comprising fennel, ginger and lemon balm for reducing one or more (pre)menstrual symptoms. The composition may be a product comprising fennel, ginger and lemon balm as provided herein.

In one aspect, the menstrual symptom is abdominal cramp. In one aspect, the menstrual symptom is fatigue. In one aspect, the menstrual symptom is mood swings. In one aspect, the menstrual symptom is bloated feeling. In one aspect, the menstrual symptom is back pains. Without wishing to be bound by theory, it is believed that a combination of fennel and chamomile and/or fennel, ginger and lemon balm is particularly advantageous because such combinations of botanicals may alleviate at least two distinct symptoms (pre)menstrual symptoms.

### Administration

The composition for use according to the present invention may be administered by any suitable method. Preferably, the composition is for oral administration.

The composition for use in treating and/or preventing pre-menstrual symptoms may be administered prior to onset of menses. Preferably, the composition for use in treating and/or preventing pre-menstrual symptoms is a composition comprising fennel and chamomile as defined herein.

The composition may be administered from 3-7 days prior to onset of menses. For example, the composition may be administered for 3-7 days prior to onset of menses. Suitably, the composition is administered daily for 3-7 days prior to onset of menses. Suitably, the composition is administered daily for 3, 4, 5, 6 or 7 days prior to onset of menses. Suitably, the composition is administered daily for 5 days prior to onset of menses.

The composition for use in treating and/or preventing menstrual symptoms may be administered following onset of menses. Preferably, the composition for use in treating and/or preventing menstrual symptoms is a composition comprising fennel, ginger and lemon balm as defined herein.

The composition may be administered from 3-7 days following onset of menses. For example, the composition may be administered for 3-7 days following onset of menses. Suitably, the composition is administered daily for 3-7 days following onset of menses. Suitably, the composition is administered daily for 3, 4, 5, 6 or 7 days following onset of menses. Suitably, the composition is administered daily for 5 days following onset of menses. The composition may initially be administered on the day of or the day following onset of menses.

The composition for use in treating and/or preventing pre-menstrual symptoms and the composition for use in treating and/or preventing menstrual symptoms may be used in combination. In particular, the composition for use in treating and/or preventing pre-menstrual symptoms may be administered prior to onset of menses followed by administration of the composition for use in treating and/or preventing menstrual symptoms following onset of menses.

The composition comprising fennel and chamomile for use in treating and/or preventing pre-menstrual symptoms and/or the composition comprising fennel, ginger and lemon balm for use in treating and/or preventing menstrual symptoms may be administered as described herein.

The composition or combination of compositions may be administered for at least one menstrual cycle. The composition or combination of compositions may be administered for at least one, at least two, at least three, at least four, at least five, at least six, at least seven, at least eight, at least nine, at least ten, at least eleven or at least 12 menstrual cycles.

### Subject

The subject according to the invention is a female with an active menstrual cycle. Preferably the subject is human.

The subject may be 15-65, 15-55, 15-45 or 15-35 years of age.

### Product

In one aspect the present invention provides a product comprising fennel and chamomile.

In another aspect the present invention provides a product comprising fennel, ginger and lemon balm.

The product may be a confectionery product (e.g. a confectionery bite, a bar, a chocolate), a powder for dissolving in a beverage, or a beverage.

Preferably, the product is a confectionery product

The product of may comprise about 0.05 g-2.0 g, 0.05 g-1.0 g or 0.05 g-0.25 g fennel extract or dried fennel powder. The product may comprise 0.05 g-2.0 g, 0.05 g-1.0 g, 0.1 g-1.0 g, 0.1-0.5 g, 0.1-0.4 g, or 0.1-0.3 g fennel extract or dried fennel powder. The product may comprise 0.05 g-0.50 g, 0.05 g-0.40 g, 0.10 g-0.40 g, 0.20 g-0.30 g, or 0.10 g-0.20 g fennel extract or dried fennel powder. The product may comprise 0.05 g-0.25 g, 0.05 g-0.20 g, or 0.10 g-0.20 g fennel extract or dried fennel powder. The product may comprise about 0.10 g, about 0.15 g or about 0.20 g fennel extract or dried fennel powder. The product may comprise about 0.10 g, about 0.11 g, about 0.12 g, about 0.13 g, about 0.14 g, about 0.15 g, about 0.16 g, about 0.17 g, about 0.18 g, about 0.19 g, about 0.20 g fennel extract or dried fennel powder. The product may comprise about 0.125 g fennel extract or dried fennel powder. Preferably, the fennel is fennel extract.

The product may comprise about 0.1 g-5.0 g chamomile extract or dried chamomile powder. Suitably, the product may comprise 0.1 g-5.0 g, 0.1 g-4.0 g, 0.2-4.0 g, 0.5-4.0 g, 0.5-3.0 g, 0.5-2.0 g or 0.5-1.0 g chamomile extract or dried chamomile powder. Suitably, the product may comprise about 0.5 g to about 2.0 g of chamomile extract or dried chamomile powder. Suitably, the product may comprise about 0.50 g, about 0.60 g, about 0.70 g, about 0.80 g, about 0.90 g, about 1.0 g of chamomile extract or dried chamomile powder. Suitably, product may comprise about 0.85 g chamomile extract or dried chamomile powder. Suitably, product may comprise about 0.425 g chamomile extract or dried chamomile powder. Preferably, the chamomile is chamomile extract.

The product may comprise about 0.1 g-5.0 g ginger extract or dried ginger powder. Suitably, the product may comprise 0.1 g-5.0 g, 0.1 g-4.0 g, 0.2-4.0 g, 0.5-4.0 g, 0.5-3.0 g, 0.5-2.0 g or 0.5-1.0 g ginger extract or dried ginger powder. Suitably, the product may comprise about 0.5 g to about 2.0 g of ginger extract or dried ginger powder. Suitably, the product may comprise about 0.50 g, about 0.60 g, about 0.70 g, about 0.80 g, about 0.90 g, about 1.0 g of ginger extract or dried ginger powder. Suitably, product may comprise about 0.85 g ginger extract or dried ginger powder. Suitably, product may comprise about 0.45 g ginger extract or dried ginger powder. Preferably, the ginger is ginger extract.

The product may comprise about 0.1 g-5.0 g lemon balm extract or dried lemon balm powder. Suitably, the product may comprise 0.1 g-5.0 g, 0.1 g-4.0 g, 0.2-4.0 g, 0.5-4.0 g, 0.5-3.0 g, 0.5-2.0 g or 0.5-1.0 g lemon balm extract or dried lemon balm powder. Suitably, the product may comprise about 0.5 g to about 2.0 g of lemon balm extract or dried lemon balm powder. Suitably, the product may comprise about 0.50 g, about 0.60 g, about 0.70 g, about 0.80 g, about 0.90 g, about 1.0 g of lemon balm extract or dried lemon balm powder. Suitably, product may comprise about 0.85 g lemon balm or dried lemon balm powder. Suitably, product may comprise about 0.45 g lemon balm extract or dried lemon balm powder. Preferably, the lemon balm is lemon balm extract.

The product may comprise about 10-460 mg, 10-400 mg, 10-300 mg, 25-300 mg, 25-200 mg, 25-200 mg, 50-200 mg, 25-100 mg, or 50-100mg magnesium. Suitably, the product may comprise about 35 mg, about 50 mg, about 70 mg or about 100 mg magnesium. Suitably, product may comprise about 70 mg magnesium. Suitably, product may comprise about 35 mg magnesium.

The product may comprise about 0.1-0.3 g fennel extract or dried fennel powder and about 0.5-1.0 g chamomile extract or dried chamomile powder. The product may comprise about 0.1-0.3 g fennel extract or dried fennel powder, about 0.5-2.0 g chamomile extract or dried chamomile powder and about 35-100 mg magnesium. The product may comprise about 0.125 g fennel extract or dried fennel powder, about 0.425 g chamomile extract or dried chamomile powder and about 35 mg magnesium.

The product may comprise about 0.1-0.3 g fennel extract or dried fennel powder, about 0.25 g-1.0 g ginger extract or dried ginger powder and about 0.25 g-1.0 g lemon balm extract or dried lemon balm powder. The product may comprise about 0.125 g fennel extract or dried fennel powder, about 0.45 g ginger extract or dried ginger powder and about 0.45 g lemon balm extract or dried lemon balm powder.

The product may comprise iron. For example, the product may comprise about 1-20 mg, 1-15 mg, 1-10 mg, 1-5 mg, 1-3mg iron. Suitably, the product may comprise about 1 mg, about 2 mg, or about 3 mg iron. Suitably, the product may comprise about 2.0 mg, about 2.2 mg, about 2.4 mg, about 2.6 mg, about 2.8 mg or about 3.0 mg iron. Suitably, the product may comprise about 2.6 mg or about 2.8 mg iron.

Preferably, the product is a confectionery product.

The confectionery product may be a confectionery bite. As used herein, a confectionary bite refers to a confectionery that is intended or suitable for consumption in one or two bites. For example, a confectionery bite may be similar in size to a truffle or brigadeiro.

Suitably, the confectionery bite may be a spherical, circular, square, rectangular or star shape. Suitably, the confectionery bite is spherical.

The confectionery product may be provided in any suitable confectionery form. The confectionery product may be a solid confectionery mass.

Suitably, the product (e.g. the confectionery product) has a water activity of less than 0.6. Water activity (a_{w}) is the partial vapour pressure of water in a solution divided by the standard state partial vapour pressure of water. In the field of food science, the standard state is most often defined as the partial vapour pressure of pure water at the same temperature. Using this definition, pure distilled water has a water activity of exactly one. Accordingly, the confectionery product may have a water activity of less than 0.6 at a temperature wherein pure distilled water has a water activity of exactly one. The water activity may be determined at room temperature, for example about 20° C. to about 22° C. The water activity may be determined using methods which are known in the art, for example using a resistive electrolytic, a capacitance or a dew point hygrometer.

The product (e.g. the confectionery product) may comprise cocoa and/or fruit flavouring.

The cocoa may be in the form of cocoa powder.

The fruit flavouring may be raspberry, passion fruit, orange, cranberry, banana, or strawberry fruit flavouring. The fruit flavouring may be in the form of fruit powder.

The product, such as the confectionery product, may comprise the combinations of botanicals described herein in a matrix with the cocoa and fruit flavouring. The product, such as the confectionery product, may comprise a dough comprising cocoa and/or fruit flavouring in combination with the botanicals described herein.

Without wishing to be bound by theory, it is believed that the provision of the botanicals in such a food matrix (e.g. a matrix comprising the botanicals in combination with lipids and sugar) may provide advantageous effects in terms of the efficacy and/or bioavailability of the botanicals. This may be further enhanced by the inclusion of magnesium and/or iron, for example.

The composition or product, for example the confectionery product, may provide about 50-500 kcal, about 50-300 kcal, about 50-200 kcal, about 100-200 kcal or about 150-200 kcal per portion. Preferably, the composition or product may provide about 150 to about 200 kcal per portion.

The composition or product, for example the confectionery product, may comprise about 5-20 g, about 5 -15 g, about 5-12 g, about 7-12 g or about 7-10 g total fat per portion. Preferably, the composition or product may comprise about 7 g to about 10 g total fat per portion.

The composition or product, for example the confectionery product, may comprise about 0.25-5 g, about 0.25-3 g, about 0.5-3 g, about 1-3 g, about 1.5-3 g or about 1.5-2 g saturated fat per portion. Preferably, the composition or product may comprise about 1.5 g to about 2 g saturated fat per portion.

The composition or product, for example the confectionery product, may comprise about 5-100 g, about 5 -75 g, about 5-50 g, about 10-50 g, about 10-25 g, or about 15-25 g total carbohydrates per portion. Preferably, the composition or product may comprise about 15 g to about 25 g total carbohydrates per portion.

The composition or product, for example the confectionery product, may comprise about 0.5-10 g, about 0.5-7.5 g, about 2.5-7.5 g, or about 4-6 g dietary fibre per portion. Preferably, the composition or product may comprise about 4 g to about 6 g dietary fibre per portion.

The composition or product, for example the confectionery product, may comprise about 2.5-50 g, about 2.5-25 g, about 5 -25 g, about 5 -15 g, or about 10-15 g total sugar per portion. Preferably, the composition or product may comprise about 10 g to about 15 g total sugar per portion.

The composition or product, for example the confectionery product, may comprise about 0.5-20 g, about 0.5-10 g, about 1-10 g, about 1-7.5 g, about 2-7.5 g or about 2-5 g protein per portion. Preferably, the composition or product may comprise about 2 g to about 5 g protein per portion.

The composition or product, for example the confectionery product, may comprise about 10-200 mg, about 10-150 mg, about 10-100 mg, about 25-100 mg or about 25-75 mg calcium per portion. Preferably, the composition or product may comprise about 25 mg to about 75 mg calcium per portion.

The composition or product, for example the confectionery product, may comprise about 50-1000 mg, about 50 -750 mg, about 50-500 mg, about 100-500 mg, about 100-300 mg, or about 200-300 mg potassium per portion. Preferably, the composition or product may comprise about 200 mg to about 300 mg potassium per portion.

Suitably, the composition or product (e.g. the confectionery product) comprises essentially no trans-fatty acid, cholesterol, sodium and/or vitamin D.

Preferably, the composition or product (e.g. the confectionery product) comprises essentially no trans-fatty acid, essentially no cholesterol, essentially no sodium and essentially no vitamin D.

The present invention also provides a combined packaging comprising a first product comprising fennel and chamomile and a second product comprising fennel, ginger and lemon balm.

The first product comprising fennel and chamomile may be a confectionary comprising fennel and chamomile as provided in the embodiments described herein. The second product comprising fennel, ginger and lemon balm may be a product comprising fennel, ginger and lemon balm as provided in the embodiments described herein.

The combined packaging may be referred to as a ‘kit’.

The kit may comprise 1-20, 1-15, 3-15, 3-10, 3-7, 5-15, 5-10 or 5-7 portions of the first product. The kit may comprise 3-7 portions of the first product. The kit may comprise 3, 4, 5, 6 or 7 portions of the first product.

The kit may comprise 1-20, 1-15, 3-15, 3-10, 3-7, 5-15, 5-10 or 5-7 portions of the second product. The kit may comprise 3-7 portions of the second product. The kit may comprise 3, 4, 5, 6 or 7 portions of the second product.

The kit may comprise 3-7 portions of the first product and 3-7 portions of the second product.

The kit may comprise 5 portions of the first product and 5 portions of the second product.

Suitably, each portion is for daily consumption. Suitably, each portion is for daily consumption and provides a daily dosage of one or more botanical as provided by the composition for use according to the present invention.

Each portion may be individually packaged within the kit. Multiple portions may be packaged together within the kit.

### Method of Manufacture

The present invention also provides a process for the preparation of a confectionery product according to the invention, comprising the steps of:
a) providing a powder mix comprising (i) fennel extract powder or dried fennel powder and chamomile extract powder or dried chamomile powder; or (ii) fennel extract powder or dried fennel powder, ginger extract powder or dried ginger powder and lemon balm extract powder or dried lemon balm powder;
b) combining the powder mix with a dough mix and mixing to form a combined dough;
c) moulding or shaping the combined dough to form a confectionery mass.

The powder mix may further comprise cocoa, in particular cocoa powder, and/or one or more fruit flavourings.

The fruit flavouring may be selected from raspberry, passion fruit, orange, cranberry, banana, or strawberry. The fruit flavouring may be a fruit powder.

The dough mix may comprise almond butter, dates syrup and/or crushed almond.

The method may comprise combining a paste, for example a date paste, with the powder mix and dough mix prior to mixing to form a combined dough.

The date paste may be warmed to about 35° C. prior to combining with the powder mix and dough mix.

Mixing may be performed in any apparatus suitable for mixing a dough.

The present method thus produces a combined dough comprising the powder mix and dough mix in a food matrix.

Following mixing, the combined dough may be cooled to less than about 25° C.

The cooled dough may then be subject to a moulding/filling process to produce confectionery bites. Suitable apparatus for such moulding/filling include a CORNUCOPIA® KN135 (Rhéon).

Optionally, further ingredients (e.g. almond butter) may be added to the combined dough produced by mixing the powder mix and dough mix. The further ingredients may be added prior to moulding/filling.

Optionally a coating, for example coconut flake coating, may be applied to the confectionery bites.

### EMBODIMENTS

The present invention provides the aspects according to the following numbered clauses:
1. A composition for use in treating and/or preventing menstrual symptoms wherein the composition comprises fennel and chamomile.
2. The composition for use according to clause 1 wherein the fennel is fennel extract or dried fennel powder.
3. The composition for use according to clause 1 or 2 wherein the composition is administered to a subject to provide a daily dosage of 0.05 g-2.0 g fennel extract or dried fennel powder, preferably about 0.25 g/day fennel extract or dried fennel powder.
4. The composition for use according to any preceding clause wherein the chamomile is a chamomile extract or dried chamomile powder.
5. The composition for use according to clause 4 wherein the composition is administered to a subject to provide a daily dosage of 0.1 g-5.0 g chamomile extract or dried chamomile powder, preferably about 0.85 g/day chamomile extract or dried chamomile powder.
6. The composition for use according to any preceding clause wherein the composition further comprises magnesium.
7. The composition for use according to clause 6 wherein the composition is administered to a subject to provide a daily dosage of 10-460 mg magnesium, preferably about 70 mg/day magnesium.
8. The composition for use according to any preceding clause wherein the menstrual symptoms are pre-menstrual symptoms, selected from one or more of fatigue, mood swings, binge eating or cravings, headache, and/or abdominal cramp.
9. The composition for use according to any preceding clause wherein the composition is administered prior to onset of menses, preferably for 3-7 days prior to onset of menses.
10. The composition for use according to clause 9 wherein the composition is administered for 5 days prior to the onset of menses.
11. The composition for use according to any preceding clause wherein the composition is a confectionery product.
12. The composition for use according to clause 11 wherein the confectionery product has a water activity of less than 0.6.
13. The composition for use according to clause 11 or 12 wherein the confectionery product is a confectionery bite.
14. The composition for use according to any of clauses 11 to 13 wherein the confectionery product comprises cocoa and/or fruit flavouring.
15. A composition for use in treating and/or preventing menstrual symptoms wherein the composition comprises fennel, ginger and lemon balm, wherein the menstrual symptoms are selected from one or more of abdominal cramp, fatigue, mood swings, bloated feeling, and/or back pains.
16. The composition for use according to clause 15 wherein the fennel is fennel extract or dried fennel powder.
17. The composition for use according to clause 16 wherein the composition is administered to a subject to provide a daily dosage of 0.05 g-2.0 g fennel extract or dried fennel powder, preferably about 0.25 g/day fennel extract.
18. The composition for use according to any of clauses 15 to 17 wherein the ginger is ginger extract or dried ginger powder.
19. The composition for use according to clause 14 wherein the composition is administered to a subject to provide a daily dosage of 0.1 g-5.0 g ginger extract or dried ginger powder, preferably about 0.9 g/day ginger extract or dried ginger powder.
20. The composition for use according to any of clauses 15 to 19 wherein the lemon balm is lemon balm extract or dried lemon balm powder.
21. The composition for use according to clause 20 wherein the composition is administered to a subject to provide a daily dosage of 0.1 g-5.0 g lemon balm extract or dried lemon balm powder, preferably about 0.9 g/day lemon balm extract or dried lemon balm powder.
22. The composition for use according to any of clauses 15 to 21 wherein the composition is administered after the onset of menses, preferably for 3-7 days after the onset of menses.
23. The composition for use according to clause 22 wherein the composition is administered for 5 days after the onset of menses.
24. The composition for use according to any of clauses 15 to 23 wherein the composition is a confectionery product.
25. The composition for use according to clause 24 wherein the confectionery product has a water activity of less than 0.6.
26. The composition for use according to clause 24 or 25 wherein the confectionery product is a confectionery bite.
27. The composition for use according to any of clauses 24 to 26 wherein the confectionery product comprises cocoa and/or fruit flavouring.
28. The composition for use according to any of clauses 1 to 14, wherein the composition is administered prior to the onset of menses and is followed by administration of a composition for use according to any of clauses 15 to 27 after the onset of menses.
29. The composition for use according to any of clauses 15 to 27, wherein the composition is administered after the onset of menses and following administration of the composition for use according to any of clauses 1 to 14 prior to the onset of menses.
30. A combination of a first composition comprising fennel and chamomile and a second composition comprising fennel, ginger and lemon balm for use in treating and/or preventing menstrual symptoms.
31. A combination for use according to clause 30 wherein the first composition is a composition for use according to any of clauses 1 to 14, wherein the premenstrual symptoms are selected from one or more of fatigue, mood swings, binge eating or cravings, headache, and/or abdominal cramp.
32. A combination for use according to clause 30 or 31 wherein the second composition is a composition for use according to any of clauses 15 to 27, wherein the menstrual symptoms are selected form one or more of abdominal cramp, fatigue, mood swings, bloated feeling, and/or back pains.
33. A method of treating and/or preventing menstrual symptoms comprising administering a composition comprising fennel and chamomile to a subject.
34. A method according to clause 33 wherein the composition is a composition as defined in any of clauses 1 to 7 or 11 to 14.
35. A method according to clause 33 or 34 wherein the composition is administered according to any of clauses 8 to 10.
36. A method of treating and/or preventing menstrual symptoms comprising administering a composition comprising fennel, ginger and lemon balm to a subject.
37. A method according to clause 36 wherein the composition is a composition as defined in any of clauses 15 to 21 or 24 to 27.
38. A method according to clause 36 or 37 wherein the composition is administered according to clause 22 or 23.
39. A method for treating and/or preventing menstrual symptoms comprising administering a combination of a first composition comprising fennel and chamomile and a second composition comprising fennel, ginger and lemon balm to a subject.
40. A method according to clause 39 wherein the first composition is a composition as defined in any of clauses 1 to 7 or 11 to 14.
41. A method according to clause 39 or 40 wherein the first composition is administered according to any of clauses 8 to 10.
42. A method according to any of clauses 39 to 41 wherein the second composition is a composition as defined in any of clauses 15 to 21 or 24 to 27.
43. A method according to any of clauses 39 to 42 wherein the second composition is administered according to clause 22 or 23.
44. A product comprising fennel and chamomile, preferably wherein the product is a confectionery product.
45. The product according to clause 44, wherein the fennel is fennel extract or dried fennel powder.
46. The method according to clause 43 wherein the product comprises 0.05 g-2.0 g, 0.05 g-1.0 g or 0.05 g-0.25 g fennel extract or dried fennel powder, preferably about 0.125 g fennel extract or dried fennel powder.
47. The product according to any of clause 44 wherein the chamomile is chamomile extract or dried chamomile powder.
48. The method according to clause 43 wherein the product comprises 0.19-5.0 g, 0.1 g-2.0 g or 0.25 g-0.75 g chamomile extract or dried chamomile powder, preferably about 0.425 g chamomile extract or dried chamomile powder.
49. The product according to any of clauses 44 to 48 wherein the product further comprises magnesium.
50. The product according to clause 49 wherein the product comprises 10-460 mg, 10-250 mg or 15-75 mg magnesium, preferably 35 mg magnesium.
51. The product according to any of clauses 44 to 50 wherein the product has a water activity of less than 0.6.
52. The product according to any of clauses 44 to 51 wherein the product is a confectionery bite.
53. The product according to any of clauses 44 to 52 wherein the product comprises cocoa and/or fruit flavouring.
54. A product comprising fennel, ginger and lemon balm, preferably wherein the product is a confectionery product.
55. The product according to clause 54 wherein the fennel is fennel extract or dried fennel powder.
56. The product according to clause 55 wherein the product comprises 0.05 g-2.0 g, 0.05 g-1.0 g or 0.05 g-0.25 g fennel extract, preferably about 0.125 g fennel extract or dried fennel powder.
57. The product according to any of clauses 54 to 56 wherein the ginger is ginger extract or dried ginger powder.
58. The product according to clause 57 wherein the product comprises 0.1 g-5.0 g, 0.1 g-2.0 g or 0.25 g-1.0 g ginger extract, preferably 0.45 g ginger extract or dried ginger powder.
59. The product according to any of clauses 54 to 58 wherein the lemon balm is lemon balm extract or dried lemon balm powder.

60. The product according to clause 59 wherein the product comprises 0.1 g-5.0 g, 0.1 g-2.0 g or 0.25 g-1.0 g lemon balm extract or dried lemon balm powder, preferably 0.45 g lemon balm extract or dried lemon balm powder.
61. The product according to any of clauses 54 to 60 wherein the product has a water activity of less than 0.6.
62. The product according to any of clauses 54 to 61 wherein the confectionery product is a confectionery bite.
63. The confectionery product according to any of clauses 54 to 62 wherein the product comprises cocoa and/or fruit flavouring.
64. A kit comprising a first product according to any of clauses 44 to 53 and a second product according to any of clauses 54 to 63.
65. A kit according to clause 64 wherein the kit comprises 3-7 portions of the first product and 3-7 portions of the second product wherein each portion is for daily consumption.
66. A kit according to clause 65 wherein a portion of the first product comprises 0.05 g-2.0 g, 0.05 g-1.0 g or 0.05 g-0.25 g fennel extract or dried fennel powder, preferably about 0.25 g fennel extract or dried fennel powder; 0.1 g-5.0 g, 0.1 g-2.0 g or 0.25 g-0.75 g chamomile extract or dried chamomile powder, preferably about 0.85 g chamomile extract or dried chamomile powder; and/or 10-460 mg, 10-250 mg or 15-75 mg magnesium, preferably about 70 mg magnesium.
67. A kit according to clause 65 or 66 wherein a portion of the second product comprises 0.05 g-2.0 g, 0.05 g-1.0 g or 0.05 g-0.25 g fennel extract or dried fennel powder, preferably about 0.25 g fennel extract or dried fennel powder; comprises 0.1 g-5.0 g, 0.1 g-2.0 g or 0.25 g-1.0 g ginger extract or dried ginger powder, preferably about 0.9 g ginger extract or dried ginger powder; and/or comprises 0.1 g-5.0 g, 0.1 g-2.0 g or 0.25 g-1.0 g lemon balm extract or dried lemon balm powder, preferably about 0.9 g lemon balm extract or dried lemon balm powder.
68. A process for the preparation of a confectionery product according to any of clauses 44 to 63, comprising the steps of:
a) providing a powder mix comprising (i) fennel extract powder or dried fennel powder and chamomile extract powder or dried chamomile powder; or (ii) fennel extract powder or dried fennel powder, ginger extract powder or dried ginger powder and lemon balm extract powder or dried lemon balm powder;
b) combining the powder mix with a dough mix and mixing to form a combined dough; and
c) moulding or shaping the combined dough to form a confectionery mass.
69. The process according to clause 68 wherein the powder mix comprises (i) fennel extract powder and chamomile extract powder; or (ii) fennel extract powder, ginger extract powder and lemon balm extract powder.
70. A confectionery product produced according to the method of clause 69.

This disclosure is not limited by the exemplary methods and materials disclosed herein, and any methods and materials similar or equivalent to those described herein can be used in the practice or testing of embodiments of this disclosure.

It must be noted that as used herein and in the appended claims, the singular forms “a”, “an”, and “the” include plural referents unless the context clearly dictates otherwise.

The terms “comprising”, “comprises” and “comprised of' as used herein are synonymous with “including”, “includes” or “containing”, “contains”, and are inclusive or open-ended and do not exclude additional, non-recited members, elements or method steps. The terms “comprising”, “comprises” and “comprised of' also include the term “consisting of'.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Botanical Extracts for Use in Treating (Pre)Menstrual Symptoms

The ability of botanical combinations to reduce premenstrual symptoms and/or menstrual pain is determined.

Two compositions are tested:
1) A premenstrual botanical composition (PBC) providing a daily dosage of 0.25 g fennel extract and 0.85 g chamomile extract; and
2) A menstrual botanical composition (MBC) providing a daily dosage of 0.25 g fennel extract, 0.9 g ginger extract and 0.9 g lemon balm extract.

Evaluation of premenstrual symptoms is performed using the Premenstrual Symptom Scoring Test (PSST). Menstrual pain is assessed using a Pain Visual Analogue Scale (PVAS). Health related quality of life is assessed via the SF36 questionnaire.

### Example 2—Use of Compositions Containing Botanical Extracts

The effect of the compositions provided in example 1 on pre-menstrual and menstrual symptoms in fifty-seven (57) women was determined.

The age range of the women was between 20 to 54. In particular, 15.9% were between ages 20 to 24, 40.9% were between ages 25 to 34, 27.3% were between ages 35 to 44, and 15.9% were between ages 45 to 54.

Respondents perceived a range of severe symptoms during premenstrual phase. The top 5 symptoms were fatigue (19%), mood swings (17%), binge eating/cravings (16%), headache (14%), and abdominal cramp (12%). On average, there were 2-3 symptoms per respondent. There was a high severity of symptoms with an average severity of 7.6 (out of 10).

Respondents also perceived a range of severe symptoms during menstrual phase. The top 5 symptoms were abdominal cramp (27%), fatigue (13%), mood swings (12%), bloated feeling (12%), and back pains (10%). On average, there were 2-3 symptoms per respondent. There was a high severity of symptoms with an average severity of 8.2 (out of 10). The symptoms were more severe than premenstrual symptoms.

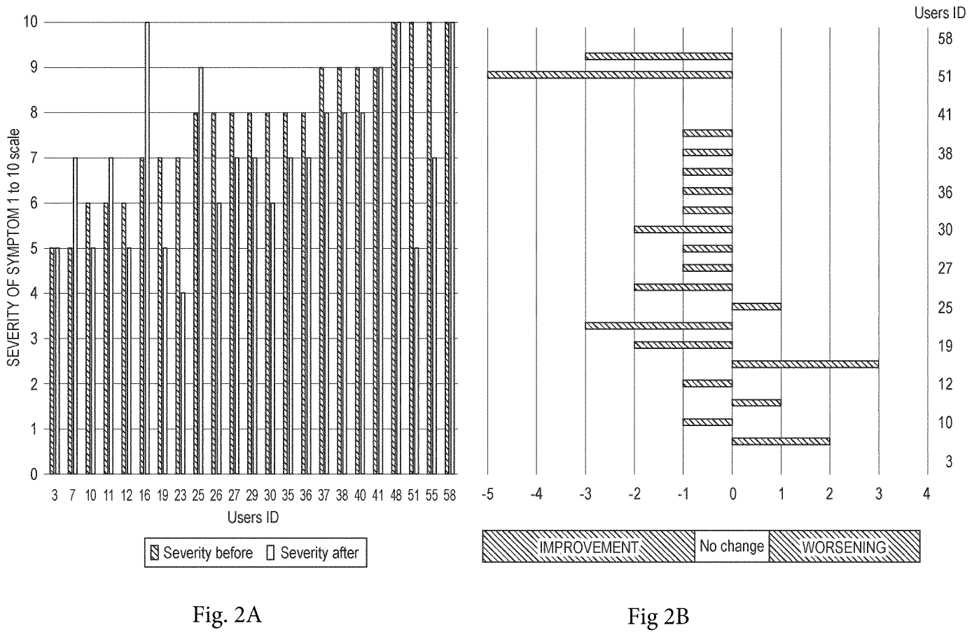

In FIGS. 2A to 7B, the left-hand graph shows the severity of different symptoms during pre-menstrual phase before and after consumption of the confectionery bites. For each user ID, the severity of the symptom before (left hand bar) and severity after (right hand bar) was assessed on a scale of 1 to 10. An assessment was made of different symptoms after consumption of confectionery bites for 1 cycle (right hand graph).

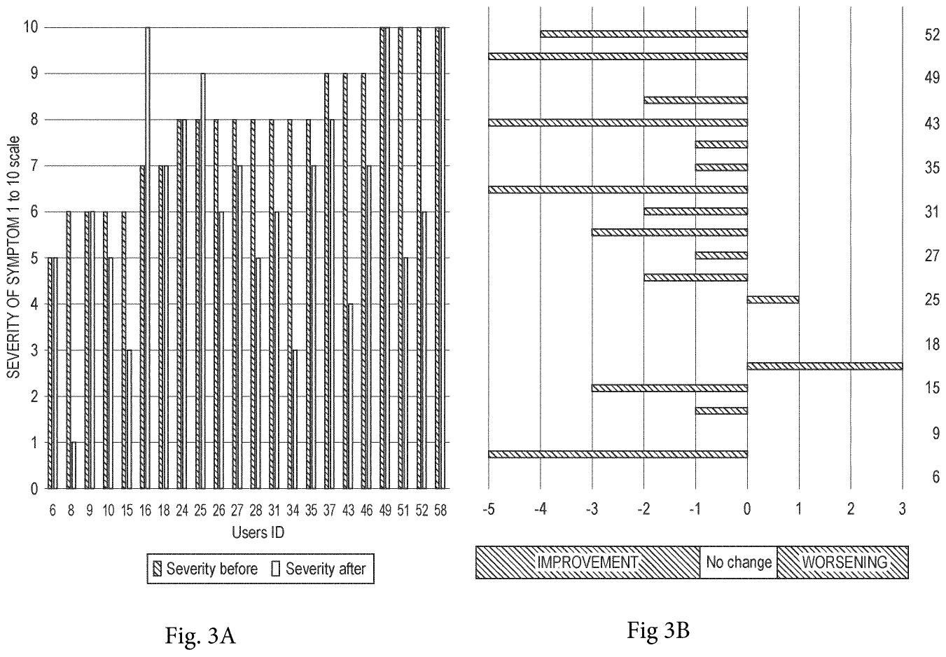

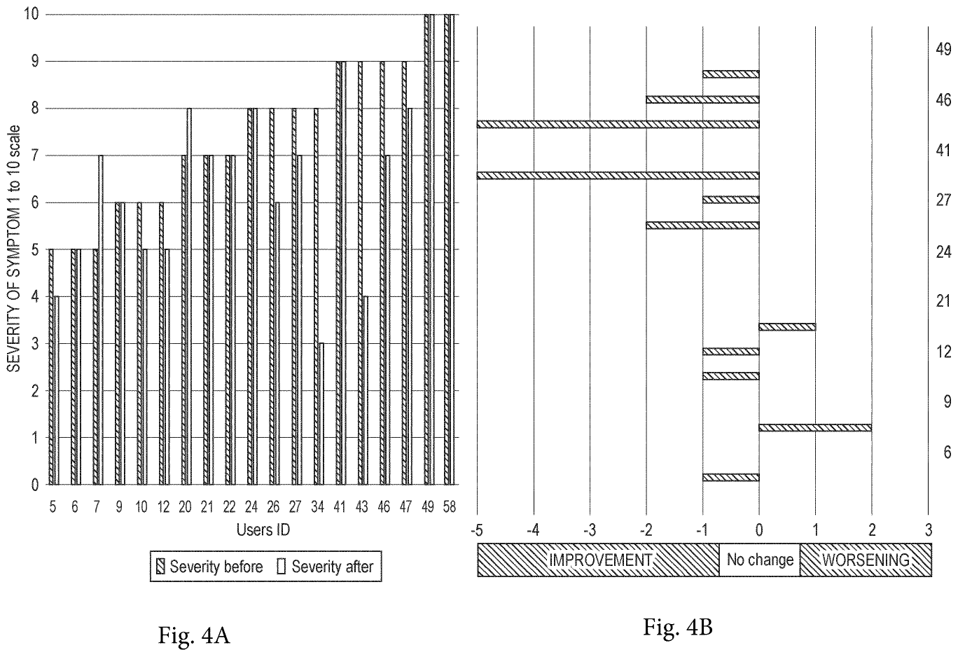

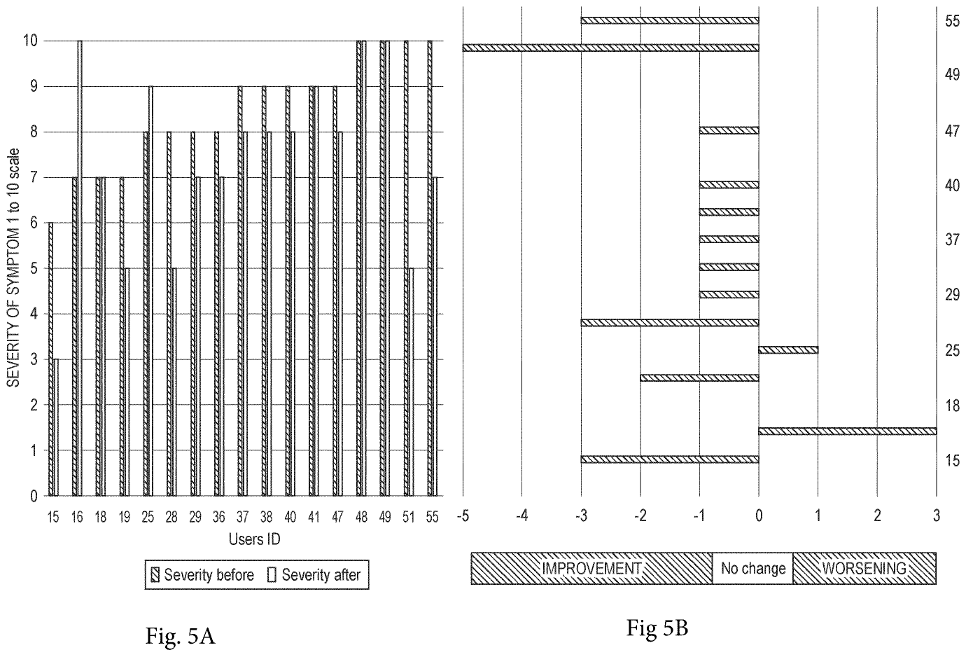

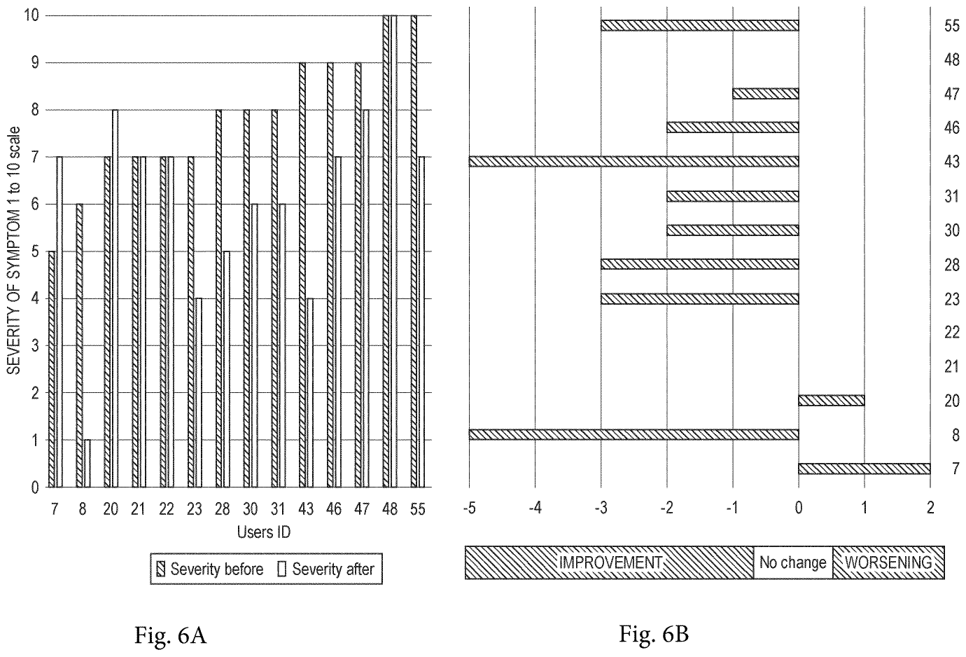

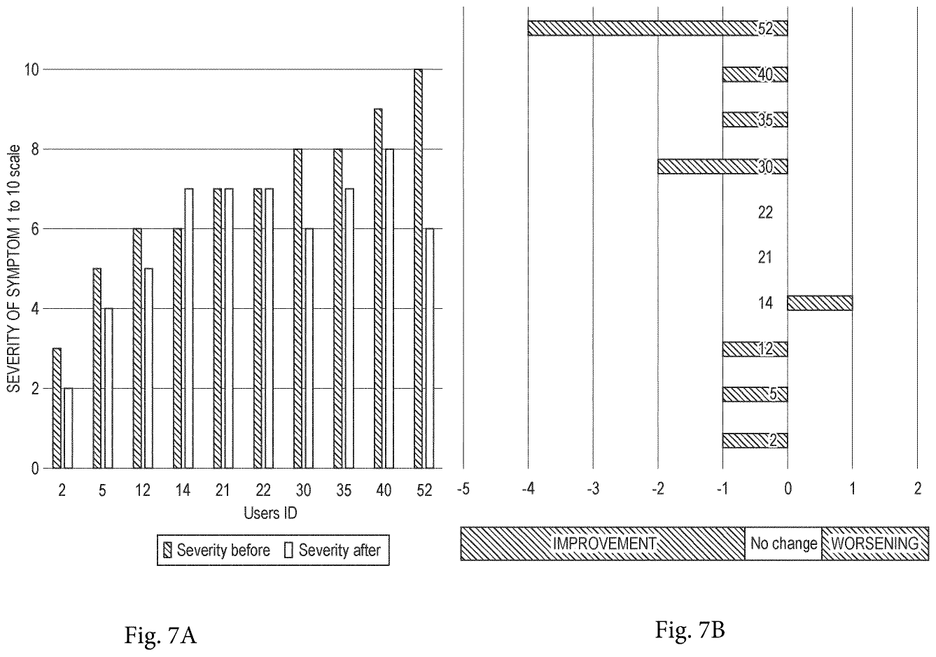

During the pre-menstrual phase, the following symptoms were most improved (in order): fatigue (FIGS. 2A-2B), mood swings (FIGS. 3A-3B), binge eating/cravings (FIGS. 4A-4B), headache (FIGS. 5A-5B), abdominal cramps (FIGS. 6A-6B) and bloating (FIGS. 7A-7B). Fatigue was the most improved, through to bloating as the number six symptom most improved.

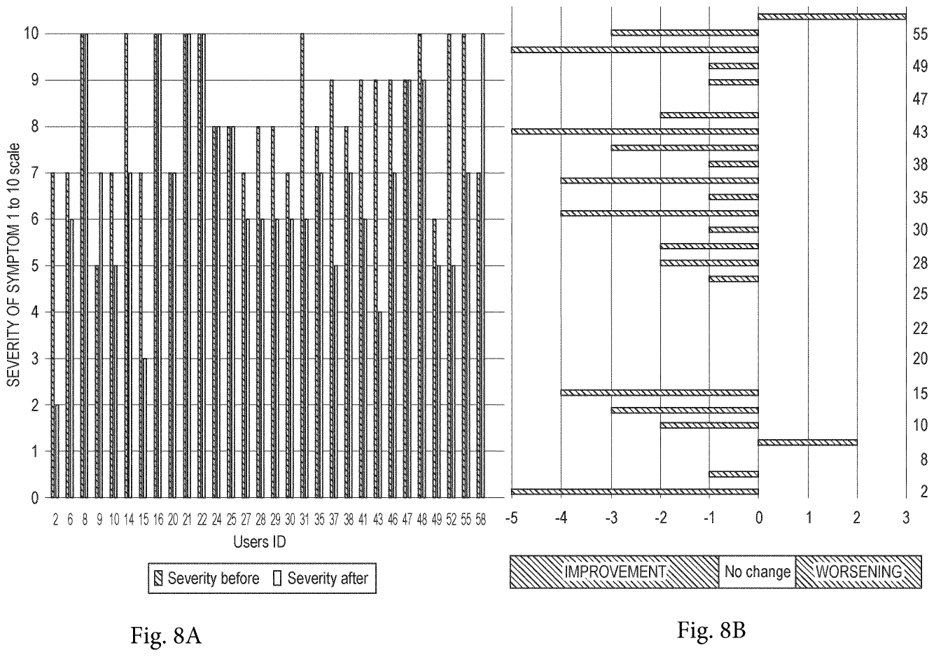

In FIGS. 8A to 13B, the left-hand graph shows the severity of different symptoms during menstrual phase before and after consumption of the confectionery bites. For each user ID, the severity of the symptom before (left hand bar) and severity after (right hand bar) was assessed on a scale of 1 to 10. An assessment was made of different symptoms after consumption of confectionery bites for 1 cycle (right hand graph).

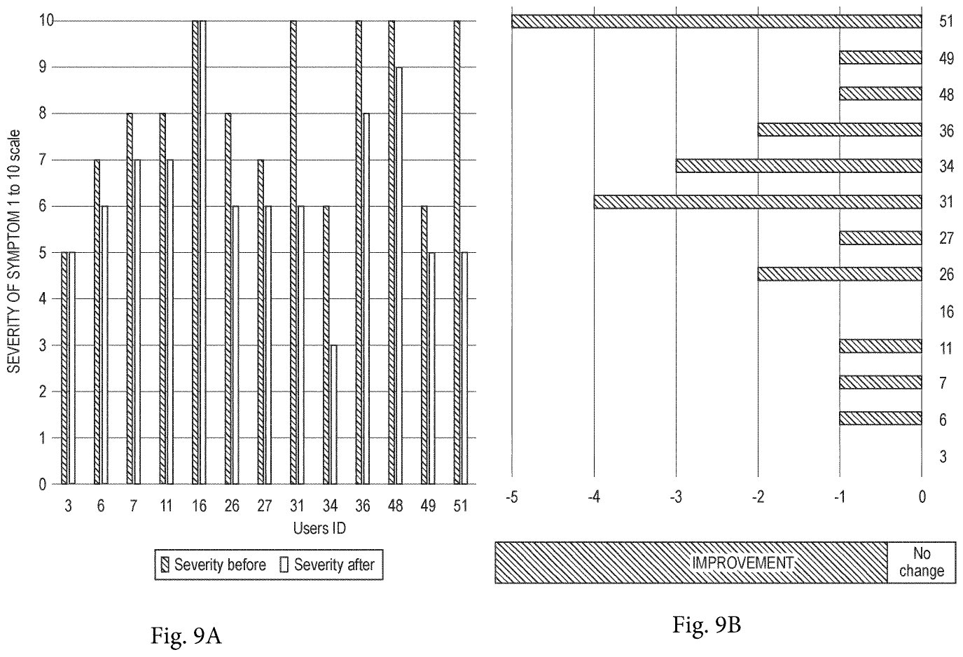

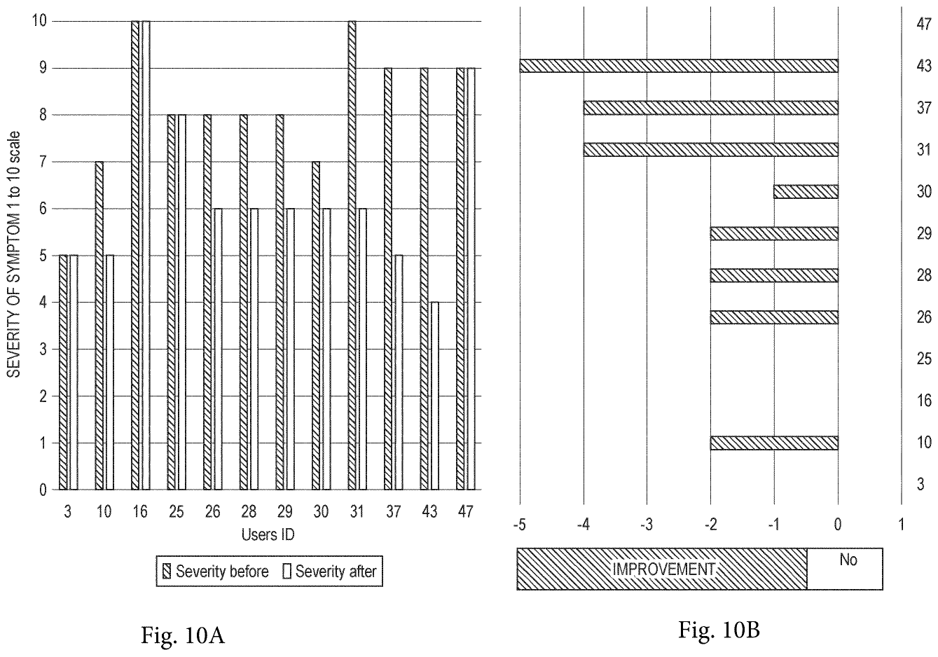

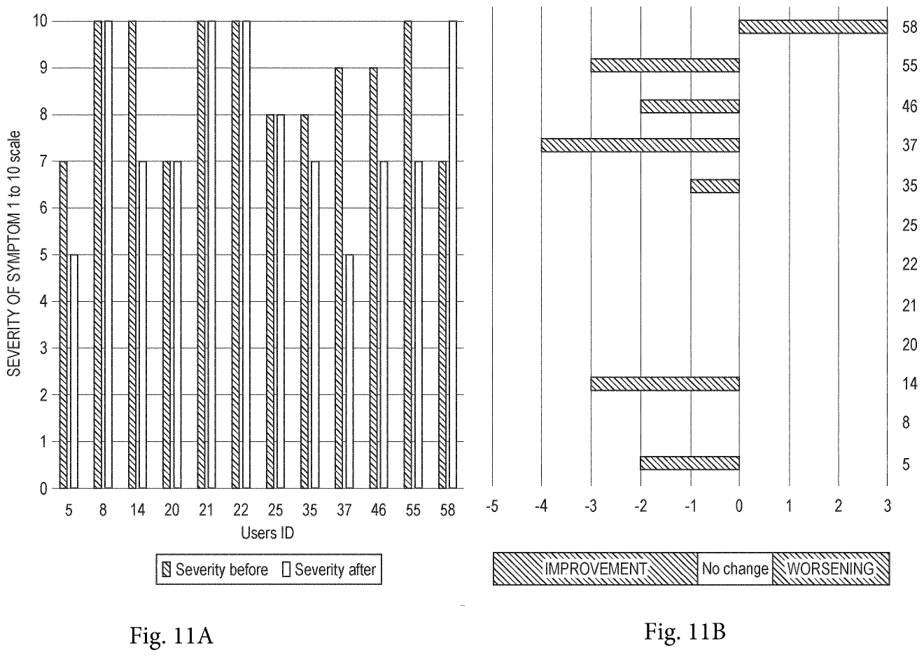

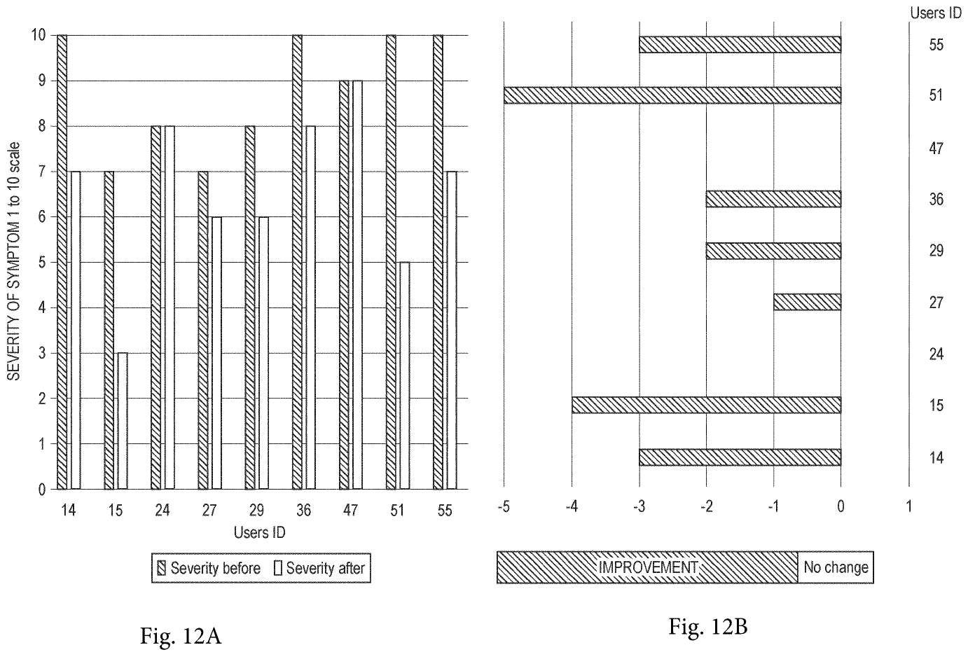

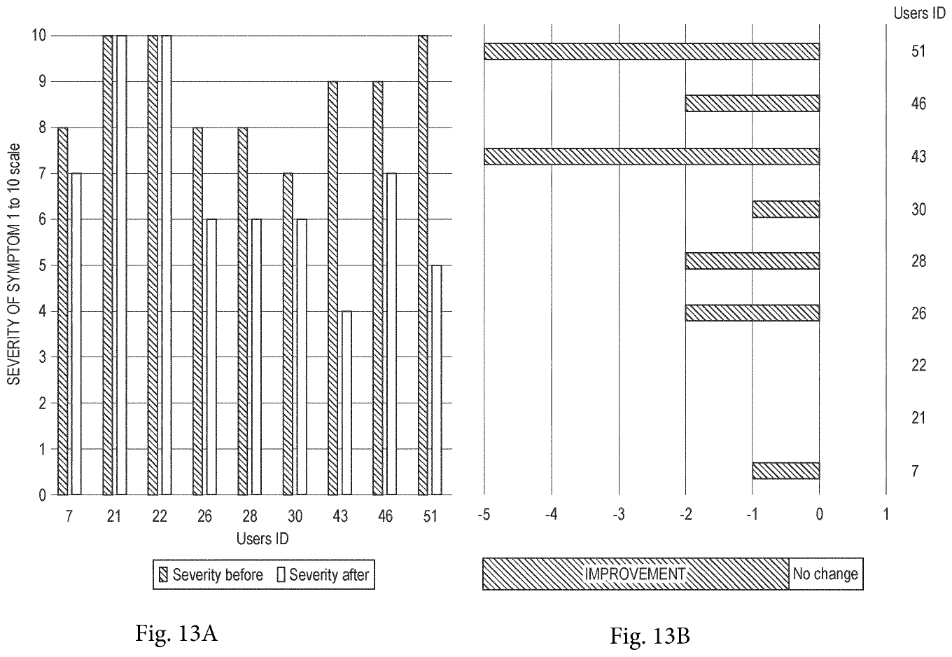

During the menstrual phase, the following symptoms were most improved (in order): abdominal cramps (FIGS. 8A-8B), fatigue (FIGS. 9A-9B), mood swings (FIGS. 10A-10B), bloating (FIGS. 11A-11B), back pain (FIGS. 12A-12B) and binge eating/cravings (FIGS. 13A-13B). Abdominal cramps was the most improved, through to binge eating/cravings as the number six symptom most improved.

### Example 3—Production of Confectionery Bites Containing Botanical Extracts

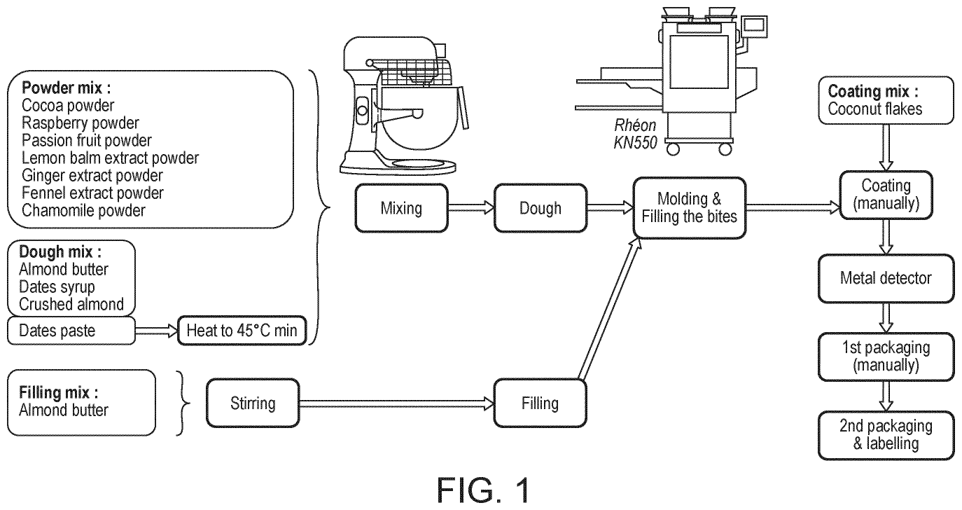

Confectionery bites containing botanicals are produced according to the schematic shown in FIG. 1.

Two confectionery bites are produced:
1) A premenstrual confectionery bite containing fennel extract and chamomile extract; and
2) A menstrual confectionery bite containing fennel extract, ginger extract and lemon balm extract.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A composition for use in treating and/or preventing menstrual symptoms wherein the composition comprises fennel and chamomile.

2. The composition for use according to claim 1 wherein:
(i) the fennel is fennel extract or dried fennel powder, wherein the composition is administered to a subject to provide a daily dosage of 0.05 g-2.0 g fennel extract or dried fennel powder, preferably about 0.25 g/day fennel extract or dried fennel powder; and
(ii) the chamomile is a chamomile extract or dried chamomile powder, wherein the composition is administered to a subject to provide a daily dosage of 0.1 g-5.0 g chamomile extract or dried chamomile powder, preferably about 0.85 g/day chamomile extract or dried chamomile powder.

3. The composition for use according to claim 1 or 2 wherein the menstrual symptoms are pre-menstrual symptoms, suitably wherein the composition is administered prior to onset of menses, preferably for 3-7 days prior to onset of menses.

4. A composition for use in treating and/or preventing menstrual symptoms wherein the composition comprises fennel, ginger and lemon balm.

5. The composition for use according to claim 4 wherein:
(i) the fennel is fennel extract or dried fennel powder, wherein the composition is administered to a subject to provide a daily dosage of 0.05 g-2.0 g fennel extract or dried fennel powder, preferably about 0.25 g/day fennel extract; and
(ii) the ginger is ginger extract or dried ginger powder, wherein the composition is administered to a subject to provide a daily dosage of 0.1 g-5.0 g ginger extract or dried ginger powder, preferably about 0.9 g/day ginger extract or dried ginger powder; and
(iii) the lemon balm is lemon balm extract or dried lemon balm powder, wherein the composition is administered to a subject to provide a daily dosage of 0.1 g-5.0 g lemon balm extract or dried lemon balm powder, preferably about 0.9 g/day lemon balm extract or dried lemon balm powder.

6. The composition for use according to claim 4 or 5 wherein the composition is administered after the onset of menses, preferably for 3-7 days after the onset of menses.

7. The composition for use according to any preceding claim wherein the composition is a confectionery product.

8. A combination of a first composition comprising fennel and chamomile and a second composition comprising fennel, ginger and lemon balm for use in treating and/or preventing menstrual symptoms.

9. A combination for use according to claim 8 wherein the first composition is a composition for use according to any of claims 1 to 3, wherein the pre-menstrual symptom is fatigue; and/or wherein the second composition is a composition for use according to any of claims 4 to 6, wherein the menstrual symptom is abdominal cramp.

10. A combination for use according to claim 9 wherein the first composition and/or the second composition is a confectionery product.

11. A product comprising fennel and chamomile, preferably wherein the product is a confectionery product, wherein:
(i) the fennel is fennel extract or dried fennel powder, wherein the product comprises 0.05 g-2.0 g, 0.05 g-1.0 g or 0.05 g-0.25 g fennel extract or dried fennel powder, preferably about 0.125 g fennel extract or dried fennel powder; and/or
(ii) the chamomile is chamomile extract, wherein the product comprises 0.1 g-5.0 g, 0.1 g-2.0 g or 0.25 g-0.75 g chamomile extract or dried chamomile powder, preferably about 0.425 g chamomile extract or dried chamomile powder.

12. A product comprising fennel, ginger and lemon balm, preferably wherein the product is a confectionery product; wherein:
(i) the fennel is fennel extract or dried fennel powder; wherein the product comprises 0.05 g-2.0 g, 0.05 g-1.0 g or 0.05 g-0.25 g fennel extract, preferably about 0.125 g fennel extract or dried fennel powder; and
(ii) the ginger is ginger extract or dried ginger powder, wherein the product comprises 0.1 g-5.0 g, 0.1 g-2.0 g or 0.25 g-1.0 g ginger extract, preferably 0.45 g ginger extract or dried ginger powder; and
(iii) the lemon balm is lemon balm extract or dried lemon balm powder; wherein the product comprises 0.1 g-5.0 g, 0.1 g-2.0 g or 0.25 g-1.0 g lemon balm extract or dried lemon balm powder, preferably 0.45 g lemon balm extract or dried lemon balm powder.

13. A kit comprising a first product according to claim 11 and a second product according to claim 12.

14. A kit according to claim 13 wherein a portion of the first product comprises 0.05 g-2.0 g, 0.05 g-1.0 g or 0.05 g-0.25 g fennel extract or dried fennel powder, preferably about 0.25 g fennel extract or dried fennel powder; 0.1 g-5.0 g, 0.1 g-2.0 g or 0.25 g-0.75 g chamomile extract or dried chamomile powder, preferably about 0.85 g chamomile extract or dried chamomile powder; and/or 10-460 mg, 10-250 mg or 15-75 mg magnesium, preferably about 70 mg magnesium; and/or
wherein a portion of the second product comprises 0.05 g-2.0 g, 0.05 g-1.0 g or 0.05 g-0.25 g fennel extract or dried fennel powder, preferably about 0.25 g fennel extract or dried fennel powder; comprises 0.1 g-5.0 g, 0.1 g-2.0 g or 0.25 g-1.0 g ginger extract or dried ginger powder, preferably about 0.9 g ginger extract or dried ginger powder; and/or comprises 0.1 g-5.0 g, 0.1 g-2.0 g or 0.25 g-1.0 g lemon balm extract or dried lemon balm powder, preferably about 0.9 g lemon balm extract or dried lemon balm powder.

15. A process for the preparation of a confectionery product according to claim 11 or 12, comprising the steps of:
a) providing a powder mix comprising (i) fennel extract powder or dried fennel powder and chamomile extract powder or dried chamomile powder; or (ii) fennel extract powder or dried fennel powder, ginger extract powder or dried ginger powder and lemon balm extract powder or dried lemon balm powder;
b) combining the powder mix with a dough mix and mixing to form a combined dough; and
c) moulding or shaping the combined dough to form a confectionery mass.
